(19) United States
(12) Patent Application Publication
(10) Pub. No.: US 2011/0256583 A1
(43) Pub. Date: Oct. 20, 2011

(54) METHOD AND MEDIUM FOR ACCELERATING TARGET ANALYTE GROWTH IN A TEST SAMPLE

(76) Inventor: Stephen C. Edberg, Longboat Key, FL (US)

(21) Appl. No.: 13/070,184

(22) Filed: Mar. 23, 2011

Related U.S. Application Data
(60) Provisional application No. 61/340,767, filed on Mar. 23, 2010.

(51) Int. Cl. C12Q 1/04; C12N 1/20; C12N 1/21

(52) U.S. Cl. 435/34; 435/252.33; 435/252.3; 435/252.8; 435/252.1

(57) ABSTRACT

A reagent mixture and testing procedure for accelerating mixing of the formulation with a specimen sample being tested by effervescing when hydrolized, thereby causing an internal “stirring” of the reagent and the specimen sample. Examples of specimen samples that can be tested include nasal swabs and lesion swabs, environmental swabs, foodstuffs, and water, to mention a few.

This application claims the benefit of U.S. Provisional Patent Application Ser. No. 61/340,767 filed Mar. 23, 2010.

### BACKGROUND OF THE INVENTION

1. Technical Information

The present method and test reagent relates to the detection of target bacteria in a biological, environmental, or food sample, and more particularly to those methods and test mixtures utilizing an ingredient that will accelerate the test procedure.

2. Background Information

The detection of the presence or absence of infectious microorganisms in humans, animals, foods, water, environmental paraphernalia and the like is an ongoing challenge. For example, the detection of the presence or absence of Staphylococcus aureus (S. aureus) can be a virulent pathogen of animals and humans. Moreover, S. aureus can cause severe food poisoning by the production of a toxin. Diseases caused by S. aureus cover a very wide clinical spectrum, from simple skin infections to life threatening infections of the bones, heart, and organs. Of particular concern is the recognition that S. aureus infection is common after surgery. S. aureus is also associated with intravenous tubing and other implants.

The bacterium S. aureus may be transmitted between healthy individuals by skin-to-skin contact, or from a commonly shared item or a surface (e.g., tanning beds, gym equipment, food handling equipment, etc.), where the transfer may be made to a subsequent person who uses the shared item or touches the surface. Of great medical concern is the recognition that healthy people entering hospitals may “carry” S. aureus (e.g., on their skin, in their noses, etc.) without any signs or symptoms that they do so. In the presence of favorable conditions (often found in, but not limited to, hospitals), the S. aureus can activate and cause serious infection. In addition, S. aureus can be a source of food poisoning often caused by a food handler contaminating the food product (e.g., meat, poultry, eggs, salads containing mayonnaise, bakery products, dairy products, etc.).

There are two categories of S. aureus, each of which is based on an individual clone's susceptibility to the methicillin class of antibiotics. The two categories of S. aureus are methicillin susceptible S. aureus (MSSA), and methicillin resistant S. aureus (MRSA, or “target analyte”). Until only a few years ago, target analyte was most commonly found in hospitals. Now, it is frequently also present in the noses, skin, etc., of people in the non-hospital community. Moreover, these target analyte bacteria are increasingly causing serious infections in the community. Target analyte is particularly serious because only very few antibiotics (e.g., vancomycin) have been shown to be uniformly effective against target analyte.

The Center for Disease Control and Prevention actively surveys for the development of methicillin resistant S. aureus. In 2000, the Society for Healthcare Epidemiology of America guidelines recommended contact isolation for patients with target analyte. In addition to the morbidity and mortality caused by target analyte, it has been estimated that each case of infection costs at least $23,000. Accordingly, many hospitals and nursing homes proactively sample patients for target analyte. [Clancy, M., Active Screening in High-Risk units is an effective and cost-avoidant method to reduce the rate of methicillin-resistant Staphylococcus aureus Infection in the hospital, Infection Control and Hospital Epidemiology 27:1009-1017, 2006].

Meyer et al. (U.S. Pat. No. 4,035,238) describes the use of a broth for the detection of S. aureus that utilizes mannitol as a source of carbon and DNA meth. Neither of these chemicals are coagulase reactive substrates.

Rambach (U.S. Pat. No. 6,548,268) employs at least two chromogenic agents in an agar medium: 5-bromo-6-chloro-indoxyl-phosphate; and 5-bromo-4-chloro-3-indoxyl glucose in the presence of desferoxamine. An individual colony hydrolyzing these substrates will produce colors that will mix with each other and will not be independent of one another.

A large number of classical culturing procedures are utilized to detect MSSA and target analyte from human, animal, food, etc., samples. They have in common a basic medium with chemical inhibitors such as 6-8% sodium chloride, potassium tellurite, and a variety of antibiotics. For example, Stevens and Jones described the use of a trehalosemannitol-phosphatase agar. [Stevens, D. L. and Jones, C., Use of trehalose-mannitolphosphatase agar to differentiate Staphylococcus epidermidis and Staphylococcus saprophyticus from other coagulase-negative staphylococci, J. of Clin. Microbiology 20:977-980, 1984]. The use of mannitol as a carbon source and salt as a selective agent into an agar known as mannitol-salt agar has been commonly used in clinical laboratories. [Baird, A. M. and W. H. Lee., Media used in the detection and enumeration of Staphylococcus aureus, Int. J. Food Microbiology 26:209-211, 1995]. Within the prior art of culturing, it is a generally accepted procedure to perform coagulase tests utilizing samples of S. aureus that are isolated in a pure culture as a required test to achieve sufficient specificity.

The procedure “S. aureus 10” [Bio Merieux, La Balme Les Grottes, France] uses an alpha-glucosidase substrate in agar to detect S. aureus. A single substrate is utilized. [Perry, J. D. et al., Evaluation of S. aureus 10, a new chromogenic agar medium for detection of Staphylococcus aureus, J. Clin. Microbiology 41:5695-5698, 2003]. A variant of this medium, which contains added antibiotics and sodium chloride, is designed to detect target analyte. [Perry et al., Development and evaluation of a chromogenic agar medium for methicillin-resistant Staphylococcus aureus, J. of Clin. Micro. 42:4519-4523, 2004].

It would be desirable to provide a test mixture and a method that can accelerate target analyte microorganism growth reactions by stirring up the test mixture reactants and the target analyte microorganisms. This stirring effect is caused by including an effervescing compound in the test mixture which compound does not take part in the analyte growth process but merely effervesces so as to stir up and mix the reactants during the growth process.

### SUMMARY OF THE INVENTION

This invention relates to a method and test mixture for accelerating metabolic reactions of target analytes, such as microorganisms, so that the reactions come to completion more quickly. The method and test mixture can be used in reactions designed to detect the presence or absence of target microorganisms in a specimen sample. It may also be used to detect the presence or absence of Escherichia coli (E. coli) in water or another specimen sample.

The method and test mixture may also be used in reactions wherein the desired result is to grow certain microorganisms. Specific microorganisms could be genetically engineered bacteria. Another could be yeast that make insulin. When these organisms are grown in the test mixture of this invention the reaction would be accelerated and the desired final product would be available much sooner than it would be in the absence of present test mixture.

The method and test mixture can be used to in reactions wherein the desired result is to detect the presence or absence of a specific target bacteria. For example, the method and test mixture could be used in a reaction wherein the specific detection of MRSA bacteria in a first generation biological, environmental, or food sample is accomplished. In the detection of MRSA, a test mixture (or “medium”) is utilized. The medium will include growth inhibitors which will inhibit growth of MSSA but will not inhibit growth of MRSA. The medium will also include a pH control and a nutrient indicator, or a specifically metabolizable substrate that will promote growth of MRSA and will produce a detectable signal in the test sample/medium mixture if target analyte is present in the test sample. If target analyte is not present in the test sample, no detectable signal will be produced. The detectable signal will be produced typically within 6 to 8 hours after inoculation of the medium with the sample. The presence of the effervescent compound or compounds in the test media will speed up the reaction so that the signal will be produced sooner if the target analyte is in the sample.

As noted above, MSSA growth inhibitors are included in the medium to inhibit or otherwise negatively affect MSSA bacterial growth, while not interfering with target analyte bacterial growth. The untreated sample is added to the test mixture, and the resultant inoculated test sample is incubated. The MSSA growth inhibitors can include Cefoxitin, Colistin, Aztreonam, Gentamicin, Kanamycin or Sisomycin, for example.

The test mixture is preferably prepared in a form that facilitates handling, packaging, storing, etc., of the test mixture. A dry powder that can be hydrated into liquid form is a particularly preferable embodiment of the test mixture, but the present invention is not limited to a powder form. The test mixture may assume a liquid form, or any other form (e.g., paste, gel, etc.), preferably one that can be hydrated for use.

The growth promoting constituents within the test mixture that facilitate the multiplication of and sustain S. aureus can be varied to suit the application. Those in the art will recognize that many different combinations of constituents, and varying relative amounts of the same constituents, can be used to provide the same functionality. Growth promoting constituents include sources of nitrates and proteins; materials operative to assist in the generation of nucleic acid synthesis; sources of energy for the S. aureus; sources of amino acid growth factors; and, in some embodiments, materials operable to help repair damaged target organisms. This list of growth promoting constituents does not represent all of the materials that can be beneficial within the test mixture, but does illustrate materials that are acceptable (e.g., vitamins, salts, minerals, inorganic moieties, etc.). The test mixture may include other constituents that benefit the performance of the test mixture.

Of particular utility is the use of anti-ribosomal amino glycoside antibiotics, such as, Gentamicin, Kanamycin, Tobramycin, and Sisomicin to inhibit MSSA, but not MRSA.

In most applications of the present invention, it will be desirable to utilize a test mixture that includes the following: a) an effective amount of amino acids; b) an effective amount of nitrogen sources; c) an effective amount of salts; d) an effective amount of vitamins; e) an effective amount of calcium; f) an effective amount of a hydrolyzable substrate, such as one or more sugars that can be metabolized by target analyte; and g) an effective amount of the effervescent compound.

Those skilled in the art will recognize that natural sources of such amino acids can be used rather than pure sources. The natural sources (e.g. extract of whole organisms, such as yeast) may be in mixture form or in purified form. The natural mixtures can contain varying amounts of such amino acids and vitamins. Those skilled in the art will further recognize that many different combinations of amino acids and vitamins can be used in the present invention test mixture. Effective amounts of drug ingredients which selectively inhibit the growth of MSSA in the sample are also included in the testing mixture. As noted above, in the MRSA detection procedure, examples of such suitable drug ingredients include the anti-ribosomal antibiotic gentamicin, which is active against MSSA but not MRSA. Drugs in this class can stop protein synthesis.

Those in the art will further recognize that carbon, nitrogen, trace elements, vitamins, amino acids and selective agents can be provided in many forms. Generally, it is preferred to have an amount of vitamins and amino acids within a predetermined range, but those in the art will recognize that the actual properties of each ingredient may be varied so that reduction in the amount of one ingredient can be compensated by an increase in the amount of another. This is particularly relevant when the essential amino acids, trace elements or vitamins of the microbes sought to be detected are known. Some ingredients may be provided in reduced amounts or deleted if they may be synthesized endogenously by the microorganism whose presence is to be determined. Salts may be provided as a source of ions upon dissociation.

The test mixture may be packaged in a container (e.g., a test tube, a container with a flat bottom wall, etc.) that facilitates the testing process. If the medium is prepared in a form that can be hydrated, the mixture can be hydrated with sterile water or non-sterile water.

To detect the presence of a target analyte within a sample, the sample may be obtained from a biological, environmental, or food specimen. A sample collected using a nasal swab is an example of a first generation sample that is particularly convenient to collect and test using the present invention. Once collected, the sample is inoculated into the test mixture.

The inoculated sample is incubated under conditions favorable to facilitate the multiplication of target analyte that may be present within the inoculated sample, while suppressing the multiplication of non-targets that may be present in the sample. In the case of a powdered test mixture hydrated with water, the incubation may be carried out at temperatures between about 200° C. to 350° C. The combination of sequential enzyme specificity, target analyte enhancing growth factors, and a non-target suppressing antibiotic, selectivity, provides multiple hurdles which prevent the competing non-target bacteria from being detected within the test period; e.g. 24 hours or less.

The testing paraphernalia and method can be used in hospital admissions, routinely in intensive care units, in nursing homes, with dialysis patients, with people receiving home immunosuppressive therapy, and the like. It can also be used in environmental settings (e.g., gyms, tanning salons, restaurants, etc.) where the bacteria target analyte may be transferred from a human carrier. It can also be used to test various different foods for target analyte contamination. It will be appreciated that a substantial benefit of the present method and mixture is that they may be performed/used without the need for expensive equipment or skilled medical technologists. Another substantial benefit of the present method and mixture is that they are operative to detect a relatively small amount of target analyte in the test sample; e.g., the present method and mixture has detected target analyte in samples having concentrations of target analyte as low as 100 CFU/ml.

### BRIEF DESCRIPTION OF THE DRAWINGS

Details of the invention will become more readily apparent from the following detailed description of several embodiments of the invention when taken in conjunction with the accompanying drawings in which:

FIG. 1 is a side elevational view of a test tube containing a dry test mixture which is to be used in performing the target analyte presence or absence testing procedure of this invention;

FIG. 2 is a side elevational view of a set of three test tubes of the type shown in FIG. 1 after the test mixture in each tube has been hydrated;

FIG. 3 is a side elevational view of the set of test tubes of FIG. 2 after the test has been performed on a sample specimen, wherein the sample specimen has been found to be free of target analyte; and

FIG. 4 is a side elevational view of the set of test tubes of FIG. 2 after the test has been performed on a sample specimen, wherein the sample specimen has been found to contain target analyte.

### DETAILED DESCRIPTION OF SPECIFIC EMBODIMENTS OF THE INVENTION

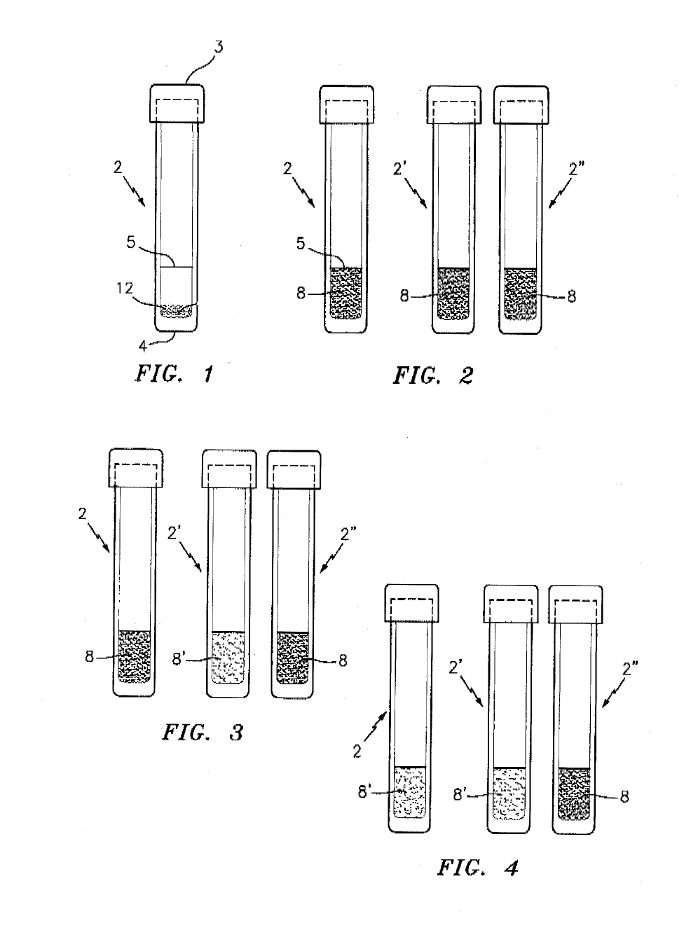

FIG. 1 is a side elevational view of a test tube denoted by the numeral 2 which contains a sample test mixture 12 for use in performing the target analyte presence/absence test of this invention. The tube 2 preferably has a flat bottom 4 and a top closure 3. The tube 2 contains a dry powdered test mixture 12 which is formed in accordance with this invention for detecting the presence or absence of target analyte in a sample; e.g., a first generational biological sample. The tube 2 is also provided with a reference line 5 which indicates the amount of hydrating liquid, preferably water, to be added to the tube 2 in order to properly hydrate the powdered mixture 12 for specimen sample testing.

An effective formulation for detecting the presence or absence of target analyte in a first generation sample of the type referred to herein is set forth below. The amounts of each ingredient in the formulation are found to be effective amounts thereof:

| | Gms/L of | | |
|---|---|---|---|
| | Test | | |
| Constituent | Mixture | Range | Function |
| MuellerHinton Broth | 8.00 | 1.0-20.0 | Protein source |
| Yeast Extract | 5.25 | 0.5-20.0 | Vitamin source |
| Lithium Chloride | 5.0 | 1.0-10.0 | Selective agent |
| Trehalose | 12.0 | 1.0-20.0 | Source of carbon |
| Phenol red | 0.018 | 0.01-0.03 | pH indicator |
| Maltose | 3.00 | 0.05-10.0 | Inducer sugar |
| Mannitol | 5.0 | 0.55-20.0 | Second carbon |
| | | | source |
| Amphotericin B | 0.005 | 0.001-0.010 | Anti-yeast sel. |
| | | | agent |
| Kinetin | 0.001 | 0.0001-0.010 | P.G. Hormone |
| Indol acetic acid | 0.005 | 0.001-0.020 | P.G. Hormone |
| Indol butyric acid | 0.005 | 0.001-0.020 | P.G. Hormone |
| Gibberellic acid | 0.001 | 0.0001-0.020 | P.G. Hormone |
| IPTG | 0.005 | 0.0001-0.020 | Inducer |
| Phenyl ethyl | 1.500 ml | 0.5 ml-10 ml | Anti-gram negative |
| alcohol | | | |
| Desfuroxime | 0.0015 | 0.0005-0.0050 | |
| Potassium | 0.500 | 0.10-5.0 | Effervescent |
| phosphate | | | |

Gentamicin and/or Tobramycin in an amount of 0.010 and in a range of 0.001-0.050 Gms/L can be used in the above test mixture formulation if the testing is for the presence or absence of MRSA in a specimen sample.

The above specific example does not represent all test mixture formulations, and the present invention is not limited thereto. As stated above, those in the art will recognize that many different combinations of constituents, and varying effective amounts of the same, can be used to provide the same functionality. Hence, the present method and mixture contemplates that a number of different constituent formulations can be made. The above mixture preferably includes an effective amount of a protein source; an effective amount of a vitamin source; an effective amount of a carbon source; an effective amount of plant hormones; an effective amount of a pH indicator; and an effective amount of an effervescent which is used to Internally stir the reagent and sample mixture during the testing procedure.

When added to the test mixture, the effervescent effervesces within the test medium, thereby stirring up the testing medium and its contents so as to accelerate the detection reaction in the medium. The effervescent thus speeds up the test so as to arrive at a definitive result sooner than if the effervescent ingredients were not included in the testing medium. An example of an acceptable effervescent is a carbon dioxide-generating combination, such as potassium phosphate and sodium bicarbonate which will cause the medium to effervesce while not significantly affecting the pH of the medium. The carbon dioxide that is produced in this preferred combination is also beneficial to the growth of certain bacteria which may be target bacteria.

FIG. 2 shows three of the test tubes 2, 2′ and 2″ wherein the powdered mixture 12 has been properly hydrated by the addition of water, preferably distilled water, to form a hydrated test mixture 8. The tube 2 is the sampling tube to which a first generation specimen sample to be analyzed for the presence or absence of MRSA is added. The specimen sample, which can be a swab of the subject being tested, is combined with the hydrated test mixture 8. The tubes 2′ and 2″ are tubes of the hydrated test mixture which are used as positive and negative controls for the test mixture. When performing the sample analysis procedure, the sample being tested is added to the hydrated test mixture 8 in the tube 2, while a sample of target analyte is added to tube 2′ and a sample of MSSA is added to tube 2″. In the instant case, when the test mixture is hydrated, the hydrated solution has a particular color which can be red for example.

As shown in the drawings, the initial color is a dark color; e.g., red. FIG. 3 shows one result of the test after a predetermined incubation period which can be from eight to twenty-four hours, for example. In the tube 2 in which the sample being analyzed was placed, there is no color change in the hydrated mixture 8, while in the positive control tube 2′ to which the target analyte was added, the color of the hydrated mixture 8′ has changed and become lighter. This is a confirmation of the presence of target analyte in the mixture 8′. It will be noted also that the hydrated test mixture 8 in the tube 2″ to which the MSSA was added did not change color. Thus the specimen tube 2 indicates no presence of target analyte while the tube 2′ does and the tube 2″ does not. Thus, this test result indicates that the specimen sample being tested is free of target analyte. FIG. 4 indicates that the specimen being tested in the tube 2 does contain target analyte since the hydrated specimen sample test mixture changes to the color 8′ which is the same as the color of the test mixture 8′ in the positive control tube 2′. This color change indicates that the specimen sample does contain target analyte.

First generational test samples can be collected by a variety of different techniques; e.g., a human sample can be collected by wiping a swab within the nose of a subject. Nasal swabs are a particularly convenient way of collecting a test sample, but they are not the only collection method; e.g., test samples can be collected from throat swabs, skin lesions, undamaged skin, etc. First generational environmental samples can be collected by various known methods for example, by wiping or swabbing a surface using a dry or wet wipe/swab. Likewise, first generational food samples can be collected from the food itself, or wiping food residue from surfaces in contact with the food, etc. Once the sample is collected, it can be deposited in the hydrated test mixture by using the same cotton swab which has been used to gather the first generation sample from the source thereof. Once the specimen sample is deposited in the test mixture, it is incubated within the test mixture for a period of time typically less than twenty-four hours. The inclusion of the effervescing component will speed up the reaction time so that results can be determined in less time than without the effervescing component. The incubation may occur at any temperature that is acceptable under the circumstances.

In addition to the above formulation, a control formulation which will rule out false positive results can be included in test kits for performing the analysis. The control formulation will be the same as that set forth above with the exception that it will not include Cefoxitin.

While the invention has been described with respect to preferred embodiments, those skilled in the art will readily appreciate that various changes and/or modifications can be made to the invention without departing from the spirit or scope of the invention as defined by appended claims.

What is claimed is:

1. A method for accelerating the growth of particular microorganisms in a reagent mixture, said method comprising the steps of:
a) providing effective amounts of growth supporting materials in said reagent mixture which materials will promote the growth of the particular microorganisms;
b) combining with the reagent mixture a specimen sample containing, or thought to contain, the particular microorganisms;
c) hydrating the reagent mixture and specimen sample combination; and
d) including an effective amount of a compound in said combination which compound will effervesce during the growth accelerating method so as to stir up said combination causing improved mixing of said materials and said specimen sample thereby speeding up the growth of the particular microorganisms in question.

2. The method of claim 1 further comprising an antibiotic compound in said mixture, which antibiotic compound will suppress growth of competing microorganisms which can consume said growth supporting materials.

3. The method of claim 1 wherein said particular microorganism is a genetically engineered microorganism.

4. The method of claim 1 wherein said particular microorganism is E. coli.

5. The method of claim 1 wherein said particular microorganism is MRSA.

6. A method for detecting the presence or absence of a target bacteria in a first generation biological, food, or environmental specimen sample, said method comprising the steps of:
a) providing a powdered reagent, said reagent containing at least one antibiotic component which will selectively inhibit the growth of non-target bacteria; one or more hydrolysable substrates which the target bacteria can metabolize and non-target bacteria cannot; and a substance that can be metabolized by the target bacteria to produce a detectable signal in said specimen sample, said reagent also containing an effervescent component which will effervesce when hydrated so as to enhance mixing of the active components of the reagent;
b) hydrating said reagent;
c) forming a mixture of said first generation specimen sample and said hydrated reagent;
d) incubating said mixture of said hydrated reagent and said specimen sample; and
e) observing said mixture to note the presence or absence of said detectable signal in said mixture.

7. A reagent for detecting the presence or absence of a target bacteria in a first generation biological, food, or environmental specimen sample, said reagent comprising:
a) an effective amount of amino acids;
b) an effective amount of nitrogen sources;
c) an effective amount of salts;
d) an effective amount of vitamins;
e) an effective amount of calcium;
f) a substance that can be metabolized by the target bacteria to produce a detectable signal in said specimen sample
g) an effective amount of protein; and
h) an effective amount of an effervescing compound which will effervesce when hydrated so as to enhance mixing of the active components in the reagent and speed up said target bacteria detection.

8. A reagent for accelerating the growth of particular microorganisms in a reagent/specimen sample mixture, said reagent comprising:
a) effective amounts of growth supporting materials which materials promote the growth of the particular microorganisms in said sample mixture; and
b) an effective amount of a compound in said sample mixture, which compound will significantly effervesce during the growth accelerating procedure so as to stir up the reagent and the specimen sample causing improved mixing of said materials and said specimen sample thereby speeding up the growth of the particular microorganisms in question.

9. The reagent of claim 8 comprising:
a) an effective amount of amino acids;
b) an effective amount of nitrogen sources;
0) an effective amount of salts;
d) an effective amount of vitamins;
e) an effective amount of calcium; and
f) an effective amount of protein.

10. The reagent of claim 9 further comprising a substance that can be metabolized by the target bacteria to produce a detectable signal in said specimen sample.
